(19) United States
(12) Patent Application Publication
(10) Pub. No.: US 2015/0232548 A1
(43) Pub. Date: Aug. 20, 2015

(54) Combination therapy with an anti-ANG2 antibody and a CD40 agonist

(71) Applicant: Hoffmann-La Roche Inc., Little Falls, NJ (US)

(72) Inventors: Christian Klein, Bonstetten (CH); Markus Thomas, Rheinfelden (DE); Alfred Zippelius, Basel (CH); Philipp Mueller, Inzlingen (DE)

(73) Assignee: Hoffmann-La Roche Inc., Little Falls, NJ (US)

(21) Appl. No.: 14/575,121

(22) Filed: Dec. 18, 2014

(30) Foreign Application Priority Data
Dec. 20, 2013 (EP) 13198753.9
Mar. 7, 2014 (EP) 14158331.0

(51) Int. Cl. C07K 16/22; C07K 16/28

(52) U.S. Cl. CPC C07K 16/22; C07K 16/2878; C07K 2317/75; C07K 2317/31; C07K 2317/56; C07K 2317/92; C07K 2317/24; A61K 2039/507

(57) ABSTRACT

The present invention relates to the combination therapy of an antibodies that binds to human angiopoietin 2 (ANG-2) with a CD40 agonist.

The present invention relates to the combination therapy of an antibody that binds to human angiopoietin 2 (ANG-2) with a CD40 agonist.

### BACKGROUND OF THE INVENTION

Angiopoietins, which play a key role in angiogenesis and blood vessel remodeling, are part of the pro-angiogenic armamentarium of growing tumors. Importantly they are one of the major factors leading to secondary resistance during anti-VEGF therapy (Saharinen, P., et al., Trends Mol Med 17 (2011) 347-362). Both angiopoietin-1 (Ang1) and angiopoietin-2 (Ang2) are Tie2-receptor ligands. While Ang1 tends to stabilize and matures blood vessel (Yancopoulos, G. D., et al., Nature 407 (2000) 242-248) Ang2 promotes tumor angiogenesis and growth by destabilizing blood vessels. Ang2 thereby opposes Ang1 in its function (Cascone, T. et al, J Clin Oncol 30 (2012) 441-444). Along this line it has been observed that blocking Ang2, but not Ang1 normalizes tumor blood vessels (Falcon, B. L., H. Hashizume, et al., Am J Pathol 175 (2009) 2159-2170) and helps to overcome acquired resistance towards anti-VEGF therapy (Chae, S. S., W. S. Kamoun, et al., Clin Cancer Res 16 (2010) 3618-3627; Thomas, M., et al. PLoS One 8 (2013) e54923).

Immunomodulatory antibodies offer an treatment approach and might be used to directly potentiate anti-tumor immune responses or as adjuvants for anti-cancer vaccines (Melero, I., et al. Nat Rev Cancer 7 (2007) 95-106). Agonistic anti-CD40 antibodies constitute one of the most effective classes of these reagents. CD40 is a cell-surface member of the tumor necrosis factor superfamily expressed on antigen presenting cells (APCs) such as dendritic cells, B cells and macrophages. Preclinical studies with anti-CD40 agonists suggest that triggering CD40 with crosslinking antibodies on antigen presenting cells (APCs) can substitute for CD4 T cell help, normally provided via CD40 ligand, and facilitate the activation as well as expansion of CD8 effector T cells (Li, F. et al, Science 333 (2011) 1030-1034). In addition CD40-activated macrophages may also exert direct tumoricidal functions (Beatty, G. L., et al. Science 331 (2011) 1612-1616); Vonderheide, R. H., et al. Oncoimmunology 2 (2013) e23033).

The immuno-suppressive tumor microenvironment poses a major hurdle for anti-tumor immunity and immunotherapeutic treatment approaches (Beatty, G. L., et al. Science 331 (2011) 1612-1616).

### SUMMARY OF THE INVENTION

The present inventors have found that anti-ANG2 antibodies enhance the efficacy of CD40 agonists to treat cancers or delay progression of a tumor or the survival of a patient afflicted with cancer e.g. with a solid tumor. The delay of progression, the longer survival as well as the potential of reduced doses with lower risk of side effects represent a major benefit for patients.

Surprisingly a treatment of tumors with anti-ANG2 antibodies in combination with CD40 agonists showed strong synergistic effect on the time to tumor progression and the time of survival (whereas a combination of anti-VEGF antibodies in combination with CD40 agonists only showed small effects).

One aspect of the invention is an antibody that binds to human angiopoietin 2 (ANG-2) wherein the antibody is administered in combination with a CD40 agonist a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).

Another aspect of the invention is a CD40 agonist wherein the a CD40 agonist is administered in combination with an antibody that binds to human angiopoietin 2 (ANG-2) a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).

In one aspect, the invention provides a method for a) treating or delaying progression of cancer, or b) prolonging the survival of a patient suffering from cancer, or c) stimulating an immune response or function, such as T cell activity (in one embodiment CD8 effector T cell activity) or macrophage activity (in one embodiment CD40-activated macrophage activity), or d) rendering a cancer susceptible for the treatment with an antibody that binds to human angiopoietin 2 (ANG-2), wherein the method comprises the step of administering an effective amount of an anti-ANG2 antibody and an effective amount of a CD40 agonist to a patient in need thereof.

In another aspect, the invention provides the use of an anti-ANG2 antibody in the manufacture of a medicament for the treatment of cancer, wherein the antibody is for administration with a CD40 agonist.

Another aspect of the invention is an antibody that binds to human angiopoietin 2 (ANG-2) in the manufacture of a medicament for

a) for use in treating or delaying progression of cancer, or

b) for use in prolonging the survival of a patient suffering from cancer, or

c) for use in stimulating an immune response or function, such as T cell activity (in one embodiment CD8 effector T cell activity) or macrophage activity (in one embodiment CD40-activated macrophage activity), or

d) for use in rendering a cancer susceptible for the treatment with an antibody that binds to human angiopoietin 2 (ANG-2);

wherein the antibody is administered with a CD40 agonist.

Another aspect of the invention is a CD40 agonist in the manufacture of a medicament for

a) for use in treating or delaying progression of cancer, or

b) for use in prolonging the survival of a patient suffering from cancer, or

c) for use in stimulating an immune response or function, such as T cell activity (in one embodiment CD8 effector T cell activity) or macrophage activity (in one embodiment CD40-activated macrophage activity), or

d) for use in rendering a cancer susceptible for the treatment with an antibody that binds to human angiopoietin 2 (ANG-2);

wherein the a CD40 agonist is administered with an antibody that binds to human angiopoietin 2 (ANG-2).

Another aspect of the invention is an antibody that binds to human angiopoietin 2 (ANG-2) in combination with a CD40 agonist a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).

Another aspect of the invention is an antibody that binds to human angiopoietin 2 (ANG-2) in combination with a CD40 agonist in the manufacture of a medicament for

a) for use in treating or delaying progression of cancer, or

b) for use in prolonging the survival of a patient suffering from cancer, or

c) for use in stimulating an immune response or function, such as T cell activity (in one embodiment CD8 effector T cell activity) or macrophage activity (in one embodiment CD40-activated macrophage activity), or

d) for use in rendering a cancer susceptible for the treatment with an antibody that binds to human angiopoietin 2 (ANG-2).

In one aspect, the invention provides a method for a) treating or delaying progression of cancer, or b) prolonging the survival of a patient suffering from cancer, or c) stimulating an immune response or function, such as T cell activity (in one embodiment CD8 effector T cell activity) or macrophage activity (in one embodiment CD40-activated macrophage activity), or d) rendering a cancer susceptible for the treatment with an antibody that binds to human angiopoietin 2 (ANG-2), wherein the method comprises the step of administering an effective amount of an anti-ANG2 antibody and an effective amount of a CD40 agonist to a patient in need thereof.

Another aspect of the invention is an antibody that binds to human angiopoietin 2 (ANG-2) and a CD40 agonist for use in combination a) for treating or delaying progression of cancer, or b) for prolonging the survival of a patient suffering from cancer, or c) for stimulating an immune response or function, such as T cell activity (in one embodiment CD8 effector T cell activity) or macrophage activity (in one embodiment CD40-activated macrophage activity), or d) for rendering a cancer susceptible for the treatment with an antibody that binds to human angiopoietin 2 (ANG-2).

Another aspect of the invention is an antibody that binds to human angiopoietin 2 (ANG-2) and a CD40 agonist for use in the manufacture of a medicament for

a) for treating or delaying progression of cancer, or

b) for prolonging the survival of a patient suffering from cancer, or

c) for stimulating an immune response or function, such as T cell activity (in one embodiment CD8 effector T cell activity) or macrophage activity (in one embodiment CD40-activated macrophage activity), or

d) for rendering a cancer susceptible for the treatment with an antibody that binds to human angiopoietin 2 (ANG-2).

Another aspect of the invention is the use of an antibody that binds to human angiopoietin 2 (ANG-2) and a CD40 agonist in combination in the manufacture of a medicament for

a) for treating or delaying progression of cancer, or

b) for prolonging the survival of a patient suffering from cancer, or

c) for stimulating an immune response or function, such as T cell activity (in one embodiment CD8 effector T cell activity) or macrophage activity (in one embodiment CD40-activated macrophage activity), or

d) for rendering a cancer susceptible for the treatment with an antibody that binds to human angiopoietin 2 (ANG-2).

In one embodiment, the anti-ANG2 antibody is a human or humanized antibody.

Preferably, the anti-ANG2 antibody specifically binds to human ANG2 with a K_{D} value of less than 1.0×10⁻⁸ mol/l, as determined by surface plasmon resonance (Biacore™).

The anti-ANG2 antibody is preferably an IgG antibody and more preferably, the anti-ANG2 antibody is of human IgG1 or IgG4 subclass.
In one preferred embodiment the anti-ANG2 antibody inhibits the interaction of human ANG-2 with TIE2 receptor with an IC50 of 15 nM or less.
In one embodiment the CD40 agonist is an agonistic CD40 antibody or an agonistic CD40L polypeptide.

In one preferred embodiment the CD40 agonist is an agonistic CD40 antibody.

In one embodiment,
i) the anti-ANG2 antibody comprises
(a) a heavy chain variable domain amino acid sequence of SEQ ID NO:1 and a light chain variable domain amino acid sequence of SEQ ID NO:2; or
(b) a heavy chain variable domain amino acid sequence of SEQ ID NO:3 and a light chain variable domain amino acid sequence of SEQ ID NO:4; and
ii) the agonistic CD40 antibody comprises
(a) a heavy chain variable domain amino acid sequence of SEQ ID NO: 5 and a light chain variable domain amino acid sequence of SEQ ID NO: 6; or
(b) a heavy chain variable domain amino acid sequence of SEQ ID NO: 7 and a light chain variable domain amino acid sequence of SEQ ID NO: 8;

In one preferred embodiment
i) the anti-ANG2 antibody comprises
a heavy chain variable domain amino acid sequence of SEQ ID NO:1 and a light chain variable domain amino acid sequence of SEQ ID NO:2; or and
ii) the agonistic CD40 antibody comprises
a heavy chain variable domain amino acid sequence of SEQ ID NO: 5 and a light chain variable domain amino acid sequence of SEQ ID NO: 6; or
In one preferred embodiment the anti-ANG2 antibody is a bispecific antibody that binds to human ANG-2 and that binds to human VEGF.

In one preferred embodiment,
i) the bispecific antibody that binds to human ANG-2 and that binds to human VEGF comprises
a heavy chain variable domain amino acid sequence of SEQ ID NO:1 and a light chain variable domain amino acid sequence of SEQ ID NO:2; and
a heavy chain variable domain amino acid sequence of SEQ ID NO:9 and a light chain variable domain amino acid sequence of SEQ ID NO:10; and
ii) the agonistic CD40 antibody comprises
a heavy chain variable domain amino acid sequence of SEQ ID NO: 5 and a light chain variable domain amino acid sequence of SEQ ID NO: 6.

In one preferred embodiment
i) the bispecific antibody that binds to human ANG-2 and that binds to human VEGF comprises the amino acid sequences of SEQ ID NO: 11, of SEQ ID NO: 12, of SEQ ID NO: 13, and of SEQ ID NO: 14; and
ii) the agonistic CD40 antibody comprises
a heavy chain variable domain amino acid sequence of SEQ ID NO: 5 and a light chain variable domain amino acid sequence of SEQ ID NO: 6.

In one preferred embodiment the cancer comprises a solid tumor.
In one preferred embodiment the cancer is colorectal cancer, ovarian cancer, glioblastoma, gastric cancer, pancreatic cancer, breast cancer, lung cancer, hepatocellular cancer.

### DESCRIPTION OF THE FIGURES

FIG. 1 In vivo anti-tumor efficacy of Anti-ANG2 antibodies (and Anti-ANG2+anti-VEGF antibodies (=Anti-VEGF/ANG2)) in combination with an agonistic CD40 antibody (delaying of progression (tumor growth) of cancer and prolonging the survival of patients treated)—The graph represents pooled data from 3 independent experiments. Mice were treated with the antibody combinations indicated in the figure.

FIGS. 2A-C In vivo anti-tumor efficacy of Anti-ANG2 antibodies (bispecific) in combination with an agonistic CD40 antibody in subcutaneous syngeneic MC38 colon carcinoma model.
2A: Tumor growth inhibition of agonistic CD40 antibody monotheraphy (unfilled circles) compared to control (filled squares).
2B: Tumor growth inhibition of bispecific ANG2/VEGF antibody monotheraphy (unfilled squares) compared to control (filled squares).
2C: Tumor growth inhibition of the combination of agonistic CD40 antibody and bispecific ANG2/VEGF antibody (unfilled triangles) compared to control (filled squares).

FIG. 3 In vivo anti-tumor efficacy of Anti-ANG2 antibodies (bispecific) in combination with an agonistic CD40 antibody in the syngeneic CT26WT model in female Balb/c mice

### DETAILED DESCRIPTION OF THE INVENTION

The present invention relates to an anti-ANG2 antibody for use in a method of treatment of cancer in a patient, the method comprising administering the anti-ANG2 antibody and a CD40 agonist to the subject, and the inventors demonstrate herein that this combination of agents results in delayed progression (tumor growth) of cancer and prolonged survival of patients treated).

Human angiopoietin-2 (ANG-2) (alternatively abbreviated with ANGPT2 or ANG2) (SEQ ID No: 15) is described in Maisonpierre, P. C., et al., Science 277 (1997) 55-60 and Cheung, A. H., et al, Genomics 48 (1998) 389-91. The angiopoietins-1 and -2 (ANG-1(SEQ ID No: 108) and ANG-2 (SEQ ID No: 107) were discovered as ligands for the Ties, a family of tyrosine kinases that is selectively expressed within the vascular endothelium. Yancopoulos, G. D., et al., Nature 407 (2000) 242-48. There are now four definitive members of the angiopoietin family. Angiopoietin-3 and -4 (Ang-3 and Ang-4) may represent widely diverged counterparts of the same gene locus in mouse and man. Kim, I., et al., FEBS Let, 443 (1999) 353-56; Kim, I., et al., J Biol Chem 274 (1999) 26523-28. ANG-1 and ANG-2 were originally identified in tissue culture experiments as agonist and antagonist, respectively (see for ANG-1: Davies, S., et al., Cell, 87 (1996) 1161-1169; and for ANG-2: Maisonpierre, P. C., et al., Science 277 (1997) 55-60). All of the known angiopoietins bind primarily to Tie2, and both Ang-1 and -2 bind to Tie2 with an affinity of 3 nM (Kd). Maisonpierre, P. C., et al., Science 277 (1997) 55-60. Ang-1 was shown to support EC survival and to promote endothelium integrity, Davis, S., et al., Cell, 87 (1996) 1161-1169; Kwak, H. J., et al., FEBS Lett 448 (1999) 249-53; Suri, C., et al., Science 282 (1998) 468-71; Thurston, G., et al., Science 286 (1999) 2511-14; Thurston, G., et al., Nat. Med. 6 (2000) 460-63, whereas ANG-2 had the opposite effect and promoted blood vessel destabilization and regression in the absence of the survival factors VEGF or basic fibroblast growth factor. Maisonpierre, P. C., et al., Science 277 (1997) 55-60. However, many studies of ANG-2 function have suggested a more complex situation. ANG-2 might be a complex regulator of vascular remodeling that plays a role in both vessel sprouting and vessel regression. Supporting such roles for ANG-2, expression analyses reveal that ANG-2 is rapidly induced, together with VEGF, in adult settings of angiogenic sprouting, whereas ANG-2 is induced in the absence of VEGF in settings of vascular regression. Holash, J., et al., Science 284 (1999) 1994-98; Holash, J., et al., Oncogene 18 (1999) 5356-62. Consistent with a context-dependent role, ANG-2 specifically binds to the same endothelial-specific receptor, Tie-2, which is activated by Ang-1, but has context-dependent effects on its activation. Maisonpierre, P. C., et al., Science 277 (1997) 55-60.

The term “human ANG2” refers to the human protein angiopoietin 2 (SEQ ID NO: 15). As used herein, “binding to human ANG2” or “specifically binding to human ANG2” or “which binds to human ANG2” or “anti-ANG2 antibody” refers to an antibody specifically binding to the human ANG2 antigen with a binding affinity of KD-value of 1.0×10⁻⁸ mol/l or lower, in one embodiment of a KD-value of 1.0×10⁻⁹ mol/l or lower. The binding affinity is determined with a standard binding assay, such as surface plasmon resonance technique (BIAcore®, GE-Healthcare Uppsala, Sweden). Thus an “antibody binding to human ANG2” as used herein refers to an antibody specifically binding to the human ANG2 antigen with a binding affinity of KD 1.0×10⁻⁸ mol/l or lower (in one embodiment 1.0×10⁻⁸ mol/l-1.0×10⁻¹³ mol/l), in on embodiment of a KD 1.0×10⁻⁹ mol/l or lower (in one embodiment 1.0×10⁻⁹ mol/l-1.0×10⁻¹³ mol/l).

Typically antibodies that bind to human ANG2 which are useful for the treatment described herein are e.g. disclosed and described in detail in WO2010/069532 (e.g. preferably anti-ANG2 antibodies <ANG-2>Ang2i_LC06, <ANG-2>Ang2i_LC07, or <ANG-2> Ang2i_LC10); in WO2011/014469 (e.g. preferably anti-ANG2 antibody H1 H685P); in US2011/150895 (e.g. anti-ANG2 antibodies SAIT-Ang-2-5, SAIT-Ang-2-6 or humanized versions thereof); in WO2009/097325 (e.g. antibody MEDI 1/5 characterized by the VL of MEDI 1 (SEQ ID No:3 in WO 2009/097325) and the VH of (MEDI 5) (SEQ ID No:7 in WO2009/097325)), in WO 2009/105269; in WO 2006/068953 or in WO 03/030833.

In one preferred embodiment the antibody that bind to human ANG2 which are useful for the treatment described herein is characterized in comprising the
(a) a heavy chain variable domain amino acid sequence of SEQ ID NO:1 and a light chain variable domain amino acid sequence of SEQ ID NO:2; or
(b) a heavy chain variable domain amino acid sequence of SEQ ID NO:3 and a light chain variable domain amino acid sequence of SEQ ID NO:4.
In one preferred embodiment the anti-ANG2 antibody is a bispecific antibody that binds to human ANG-2 and that binds to human VEGF.
Typically antibodies bispecific antibody that binds to human ANG-2 and that binds to human VEGF which are useful for the treatment described herein are e.g. disclosed and described in detail in WO2010/040508, WO 2011/117329 or WO2012/131078. Also the VH and VL of the ANG2 antibodies described in WO2010/069532; WO2011/014469; US2011/150895; WO2009/097325; WO2009/105269; WO 2006/068953 or WO 03/030833can be used within the bispecific antibody using the anti-VEGF binding arms and structures described in WO2010/040508, WO 2011/117329 or WO2012/131078.

In one preferred embodiment,
the bispecific antibody that binds to human ANG-2 and that binds to human VEGF comprises
a heavy chain variable domain amino acid sequence of SEQ ID NO:1 and a light chain variable domain amino acid sequence of SEQ ID NO:2; and
a heavy chain variable domain amino acid sequence of SEQ ID NO:9 and a light chain variable domain amino acid sequence of SEQ ID NO:10.

In one preferred embodiment
the bispecific antibody that binds to human ANG-2 and that binds to human VEGF comprises the amino acid sequences of SEQ ID NO: 11, of SEQ ID NO: 12, of SEQ ID NO: 13, and of SEQ ID NO: 14.

The CD40 antigen is a 50 kDa cell surface glycoprotein which belongs to the Tumor Necrosis Factor Receptor (TNF-R) family. (Stamenkovic et al., EMBO J. 8:1403-10 (1989).) CD40 is expressed in many normal and tumor cell types, including B lymphocytes, dendritic cells, monocytes, macrophages, thymic epithelium, endothelial cells, fibroblasts, and smooth muscle cells. (Paulie S. et al., Cancer Immunol. Immunother. 20:23-8 (1985); Banchereau J. et al., Adv. Exp. Med. & Biol. 378:79-83 (1995); Alderson M. R. et al., J. of Exp. Med. 178:669-74 (1993); Ruggiero G. et al., J. of Immunol. 156:3737-46 (1996); Hollenbaugh D. et al., J. of Exp. Med. 182:33-40 (1995); Yellin M. J. et al., J. of Leukocyte Biol. 58:209-16 (1995); and Lazaar A. L. et al., J. of Immunol. 161:3120-7 (1998).)

CD40 is expressed in all B-lymphomas and in 70% of all solid tumors. Although constitutively expressed, CD40 is up-regulated in antigen presenting cells by maturation signals, such as LPS, IL-1beta, IFN-gamma and GM-CSF.

CD40 activation plays a critical role in regulating humoral and cellular immune responses. Antigen presentation without CD40 activation can lead to tolerance, while CD40 signaling can reverse such tolerance, enhance antigen presentation by all antigen presenting cells (APCs), lead to secretion of helper cytokines and chemokines, increase co-stimulatory molecule expression and signaling, and stimulate cytolytic activity of immune cells. CD40 plays a critical role in B cell proliferation, maturation and class switching. (Foy T. M. et al., Ann. Rev. of Immunol. 14:591-617 (1996).) Disruption of the CD40 signaling pathway leads to abnormal serum immunoglobulin isotype distribution, lack of CD4+ T cell priming, and defects in secondary humoral responses. For example, the X-linked hyper-IgM syndrome is a disease associated with a mutation in the human CD40L gene, and it is characterized by the inability of affected individuals to produce antibodies other than those of the IgM isotype, indicating that the productive interaction between CD40 and CD40L is required for an effective immune response.

CD40 engagement by CD40L leads to the association of the CD40 cytoplasmic domain with TRAFs (TNF-R associated factors). (Lee H. H. et al., Proc. Natl. Acad. Sci. USA 96:1421-6 (1999); Pullen S. S. et al., Biochemistry 37:11836-45 (1998); Grammar A. C. et al., J. of Immunol. 161:1183-93 (1998); Ishida T. K. et al., Proc. Acad Acad. Sci. USA 93:9437-42 (1996); Pullen S. S. et al., J. of Biol. Chem. 274:14246-54 (1999)). The interaction with TRAFs can culminate in the activation of both NFkappa B and Jun/AP1 pathways. (Tsukamoto N. et al., Proc. Natl. Acad. Sci. USA 96:1234-9 (1999); Sutherland C. L. et al., J. of Immunol. 162:4720-30 (1999).) Depending on cell type, this signaling leads to enhanced secretion of cytokines such as IL-6 (Jeppson J. D. et al., J. of Immunol. 161:1738-42 (1998); Uejima Y. et al., Int. Arch. of Allergy & Immunol. 110:225-32, (1996), IL-8 (Gruss H. J. et al., Blood 84:2305-14 (1994); von Leoprechting A. et al., Cancer Res. 59:1287-94 (1999); Denfeld R. W. et al., Europ. J. of Immunol. 26:2329-34 (1996)), IL-12 (Cella M. et al., J. of Exp. Med. 184:747-52 (1996); Ferlin W. G. et al., Europ. J. of Immunol. 28:525-31 (1998); Armant M. et al., Europ. J. of Immunol. 26:1430-4 (1996); Koch F. et al., J. of Exp. Med. 184:741-6 (1996); Seguin R. and L. H. Kasper, J. of Infect. Diseases 179:467-74 (1999); Chaussabel D. et al., Infection & Immunity 67:1929-34 (1999)), IL-15 (Kuniyoshi J. S. et al., Cellular Immunol. 193:48-58 (1999)) and chemokines (MIP1alpha, MIP1beta, RANTES, and others) (McDyer J. F. et al., J. of Immunol. 162:3711-7 (1999); Schaniel C. et al., J. of Exp. Med. 188:451-63 (1998); Altenburg A. et al., J. of Immunol. 162:4140-7 (1999); Deckers J. G. et al., J. of the Am. Society of Nephrology 9:1187-93 (1998)), increased expression of MHC class I and II (Santos-Argumedo L. et al., Cellular Immunol. 156:272-85 (1994)), and increased expression of adhesion molecules (e.g., ICAM) (Lee H. H. et al., Proc. Natl. Acad. Sci. USA. 96:1421-6 (1999); Grousson J. et al., Archives of Dermatol. Res. 290:325-30 (1998); Katada Y. et al., Europ. J. of Immunol. 26:192-200 (1996); Mayumi M. et al., J. of Allergy & Clin. Immunol. 96:1136-44 (1995); Flores-Romo L. et al., Immunol. 79:445-51 (1993)) and costimulatory molecules (e.g., B7) (Roy M. et al., Europ. J. of Immunol. 25:596-603 (1995); Jones K. W. and C. J. Hackett, Cellular Immunol. 174:42-53 (1996); Caux C. et al., Journal of Exp. Med. 180:1263-72 (1994); Kiener P. A. et al., J. of Immunol. 155:4917-25 (1995)). Cytokines induced by CD40 engagement enhance T cell survival and activation.

In addition to enhancement of cellular and immune function, the effects of CD40 activation include: cell recruitment and differentiation by chemokines and cytokines; activation of monocytes; increased cytolytic activity of cytolytic T lymphocyte (CTL) and natural killer (NK) cells; induction of apoptosis in CD40 positive tumors; enhancement of immunogenicity of CD40 positive tumors; and tumor-specific antibody production. The role of CD40 activation in cell-mediated immune responses is also well established, and it is reviewed in: Grewal et al., Ann. Rev. of Immunol. 16:111-35 (1998); Mackey et al., J. of Leukocyte Biol. 63:418-28 (1998); and Noelle R. J., Agents & Actions—Suppl. 49:17-22 (1998).

Studies using a cross-priming model system showed that CD40 activation of APCs can replace helper T cell requirement for the generation of cytolytic T lymphocyte (CTL). (Bennett et al., Nature 393:478-480 (1998).) Evidence from CD40L deficient mice indicates a clear requirement for CD40 signaling in helper T cell priming. (Grewal I. S. et al., Science 273:1864-7 (1996); Grewal I. S. et al., Nature 378:617-20 (1995).) CD40 activation converts otherwise tolerogenic, antigen bearing B cells into competent APCs. (Buhlmann J. E. et al., Immunity 2:645-53 (1995).) CD40 activation induces maturation and differentiation of cord blood progenitors into dendritic cells. (Flores-Romo L. et al., J. of Exp. Med. 185:341-9 (1997); Mackey M. F. et al., J. of Immunol. 161:2094-8 (1998).) CD40 activation also induces differentiation of monocytes into functional dendritic cells. (Brossart P. et al., Blood 92:4238-47 (1998).) Further, CD40 activation enhances cytolytic activity of NK cells through APC-CD40 induced cytokines (Carbone E. et al., J. of Exp. Med. 185:2053-60 (1997); Martin-Fontecha A. et al., J. of Immunol. 162:5910-6 (1999).) These observations indicate that CD40 plays an essential role in the initiation and enhancement of immune responses by inducing maturation of APCs, secretion of helper cytokines, upregulation of costimulatory molecules, and enhancement of effector functions.

The critical role of CD40 signaling in the initiation and maturation of humoral and cytotoxic immune responses makes this system an ideal target for immune enhancement. Such enhancement can be particularly important for mounting effective immune responses to tumor antigens, which are generally presented to the immune system through cross-priming of activated APCs. (Huang A. Y. et al., Ciba Foundation Symp. 187:229-44 (1994); Toes R. E. M. et al., Seminars in Immunol. 10:443-8 (1998); Albert M. L. et al., Nature 392:86-9 (1998); Bennett S. R. et al., J. of Exp. Med. 186:65-70 (1997).)

Several groups have demonstrated the effectiveness of CD40 activation for antitumor responses in vitro and in vivo. (Toes R. E. M. et al., Seminars in Immunol. 10:443-8 (1998).) Two groups, using lung metastatic model of renal cell carcinoma and subcutaneous tumors by virally transformed cells, have independently demonstrated that CD40 activation can reverse tolerance to tumor-specific antigens, resulting in efficient antitumor priming of T cells. (Sotomayor E. M. et al., Nature Medicine 5:780-787 (1999); Diehl L. et al., Nature Medicine 5:774-9 (1999).) Antitumor activity in the absence of immune cells was also reported by CD40L and anti-CD40 antibody treatment in a human breast cancer line model in SCID mice. (Hirano A. et al., Blood 93:2999-3007 (1999).) CD40 activation by anti-CD40 antibody was recently shown to eradicate CD40+ and CD40− lymphoma in mouse models. (French R. R. et al., Nature Medicine 5:548-53 (1999).) Furthermore, previous studies by Glennie and co-workers conclude that signaling activity by anti-CD40 antibodies is more effective for inducing in vivo tumor clearance than other anti-surface marker antibodies capable of recruiting effectors. (Tutt A. L. et al., J. of Immunol. 161:3176-85 (1998).) Consistent with these observations, when anti-CD40 antibodies were tested for activity against CD40+ tumor cells in vivo, most but not all of the tumoricidal activity was associated with CD40 signaling rather than ADCC. (Funakoshi S. et al., J. of Immunotherapy with Emphasis on Tumor Immunol. 19:93-101 (1996).) In another study, bone marrow dendritic cells were treated ex vivo with a variety of agents, and tested for in vivo antitumor activity. These studies demonstrated that CD40L stimulated DCs were the most mature and most effective cells that mounting an antitumor response.

The essential role of CD40 in antitumor immunity has also been demonstrated by comparing responses of wild-type and CD40−/− mice to tumor vaccines. These studies show that CD40−/− mice are incapable of achieving the tumor immunity observed in normal mice. (Mackey M. F. et al., Cancer Research 57:2569-74 (1997).) In another study, splenocytes from tumor bearing mice were stimulated with tumor cells and treated with activating anti-CD40 antibodies ex vivo, and were shown to have enhanced tumor specific CTL activity. (Donepudi M. et al., Cancer Immunol. Immunother. 48:153-164 (1999).) These studies demonstrate that CD40 occupies a critical position in antitumor immunity, in both CD40 positive and negative tumors. Since CD40 is expressed in lymphomas, leukemias, multiple myeloma, a majority of carcinomas of nasopharynx, bladder, ovary, and liver, and some breast and colorectal cancers, activation of CD40 can have a broad range of clinical applications.

The “CD40 agonist” as used herein includes any moiety that agonizes the CD40/CD40L interaction. CD40 as used in this context refers preferably to human CD40, thus the CD40 agonist is preferably an agonist of human CD40. Typically these moieties will be agonistic CD40 antibodies or agonistic CD40L polypeptides. These antibodies include by way of example human antibodies, chimeric antibodies, humanized antibodies, bispecific antibodies, scFvs, and antibody fragments that specifically agonize the CD40/CD40L binding interaction. In one preferred embodiment the agonistic CD40 antibody will comprise a chimeric, fully human or humanized CD40 antibody. In another preferred embodiment the agonistic CD40 antibody will comprise a chimeric, fully human or humanized CD40 antibody.

An “agonist” combines with a receptor on a cell and initiates a reaction or activity that is similar to or the same as that initiated by a natural ligand of the receptor. An “CD40 agonist” induces any or all of, but not limited to, the following responses: B cell proliferation and/or differentiation; upregulation of intercellular adhesion via such molecules as ICAM-1, E-selectin, VC AM, and the like; secretion of pro-inflammatory cytokines such as IL-1, IL-6, IL-8, IL-12, TNF, and the like; signal transduction through the CD40 receptor by such pathways as TRAF {e.g., TRAF2 and/or TRAF3), MAP kinases such as NIK (NF-kB inducing kinase), I-kappa B kinases (IKK/.beta.), transcription factor NF-kB, Ras and the MEK/ERK pathway, the PI3K AKT pathway, the P38 MAPK pathway, and the like; transduction of an anti-apoptotic signal by such molecules as XIAP, mcl-1, bcl-x, and the like; B and/or T cell memory generation; B cell antibody production; B cell isotype switching, up-regulation of cell-surface expression of MHC Class II and CD80/86, and the like.

By agonist activity is intended an agonist activity of at least 30%, 10 35%, 40%, 45%, 50%, 60%, 70%, 75%, 80%, 85%, 90%, 95%, or 100% greater than the agonist activity induced by a negative control as measured in an assay of a B cell response.

In one preferred embodiment an CD40 agonist has an agonist activity that is at least 2-fold greater or at least 3-fold greater than the agonist activity induced by a negative control as measured in an assay of a B cell response.

Thus, for example, where the B cell response of interest is B cell proliferation, agonist activity would be induction of a level of B cell proliferation that is at least 2-fold greater or at least 3-fold greater than the level of B cell proliferation induced by a negative control.

In one embodiment, an antibody that does not bind to CD40 serves as the negative control. A substance “free of significant agonist activity” would exhibit an agonist activity of not more than about 25% greater than the agonist activity induced by a negative control, preferably not more than about 20% greater, 15% greater, 10% greater, 5% greater, 1% greater, 0.5% greater, or even not more than about 0.1% greater than the agonist activity induced by a negative control as measured in an assay of a B cell response.

An “agonistic CD40 antibody”, or “activating CD40antibody” or “agonistic or activating anti-CD40 antibody” as used herein means an antibody that binds to human CD40 and that increases one or more CD40 activities by at least about 20% when added to a cell, tissue or organism expressing CD40. In some embodiments, the antibody activates CD40 activity by at least 40%, 50%, 60%, 70%, 80%, 85%.

In some embodiments, the activating antibody is added in the presence of CD40L.

In another preferred embodiment, the agonist activity of the agonistic CD40 antibody is measured as follows:

Increase of Immunogenicity of Cell Line Jy by Anti-CD40 Antibodies

CD40 positive JIYOYE cells (ATCC CCL 87) (“Jy cells”) were cultured and maintained in RPMI medium. JIYOYE cells were incubated for 24 hours with an anti-CD40 antibody of the invention (21.4.1), or with an isotype matched antibody (anti-KLH), in complete RPMI medium. Cells were then washed and treated with 25 mg mitomycin C (Sigma)/7 ml media for 60 min. These cells were then incubated with isolated human T cells at a 1:100 ratio for 6 days at 37 deg. C. (5% CO2). T cells were then collected, washed, and the level of CTL activity determined against fresh chromium 51 (New England Nuclear, Boston, Mass.) labeled JIYOYE cells. Specific CTL activity was calculated as % specific cytolysis=(cytolysis Jy (cpm)-spontaneous cytolysis (cpm))/(total cytolysis (cpm)-spontaneous cytolysis (cpm)).

In one preferred embodiment the agonistic CD40 antibody as used herein inreases immunogenicity in CD40 positive JIYOYE cells (ATCC CCL 87) by at least 50% as measured in (above described) in vitro JIYOYE cells (ATCC CCL 87) assay.

Agonistic CD40 antibodies also named anti-CD40 activating antibodies herein can contribute to tumor eradication via several important mechanisms. Foremost among these is activation of host dendritic cells for enhanced tumor antigen processing and presentation, as well as enhanced antigen presentation or immunogenicity of CD40 positive tumor cells themselves, leading to activation of tumor specific CD4+ and CD8+ lymphocytes. Additional antitumor activity can be mediated by other immune-enhancing effects of CD40 signaling (production of chemokines and cytokines, recruitment and activation monocytes, and enhanced CTL and NK cytolytic activity), as well as direct killing of CD40+ tumors by induction of apoptosis or by stimulating a humoral response leading to ADCC. Apoptotic and dying tumor cells can also become an important source of tumor-specific antigens that are processed and presented by CD40 activated APCs.

The present invention describes an isolated antibody or antigen-binding portion thereof that binds human CD40 and acts as a CD40 agonist.

Agonistic CD40 antibodies are described e.g. Beatty et al., Science 331 (2011) 1612-1616, R. H. Vonderheide et al., J Clin Oncol 25, 876 (2007); Khalil, M, et al., Update Cancer Ther. 2007 Jun. 1; 2(2): 61-65, an agonist CD40 rat anti-mouse IgG2a mAb FGK45 as model antibody is described in S. P. Schoenberger, et al, Nature 393, 480 (1998)); the mouse cross-reactive agonistic CD40 antibody Clone 1C10 is described in Santos-Argumedo L. et al., Cell Immunol. 156 (1994) 272-285 and Heath A W et al. Eur J Immunol 24 (1994) 1828-34. Examples in clinical trials are e.g. CP-870,893 and dacetuzumab (an agonist CD40 antibody, CAS number 880486-59-9, SGN-40; humanized S2C6 antibody) (Khalil, M, et al, Update Cancer Ther. 2007 Jun. 1; 2(2): 61-65.

In one preferred embodiment the agonistic CD40 antibody is CP-870,893 which a fully human IgG2 agonistic CD40 antibody developed by Pfizer. It binds human CD40 with a KD of 3.48×10⁻¹⁰ M, but does not block binding of CD40L (see e.g., U.S. Pat. No. 7,338,660 or EP1476185 wherein CP-870,893 is described as antibody 21.4.1). CP-870,893 (antibody 21.4.1 of U.S. Pat. No. 7,338,660) is characterized by comprising (a) a heavy chain variable domain amino acid sequence of QVQLVQSGAEVKKPGASVKVSCKAS GYTFTGYYMHWVRQAPGQGLEWMGWINPDSGGTNYAQKFQGRVTMTR DTSISTAYMELNRLRSDDTAVYYCARDQPLGYCTNGVCSYFDYWGQGTL VTVSS (SEQ ID NO: 5) (which corresponds to SEQ ID NO: 42 of U.S. Pat. No. 7,338,660) (b) a light chain variable domain amino acid sequence of DIQMTQSPSSVSASVGDRVTITCRASQGIYSWLAWYQQKPGKAPNLLIYTA STLQSGVPSRFSGSGSGTDFTLTISSLQPEDFATYYCQQANIFPLTFGGGTKV EIK (SEQ ID NO: 6) (which corresponds to SEQ ID NO: 44 of U.S. Pat. No. 7,338,660); and/or having the heavy chain variable domain and light chain variable domain amino acid sequences of the antibody produced by hybridoma 21.4.1 having American Type Culture Collection (ATCC) accession number PTA-3605. Dacetuzumab and other humanized S2C6 antibodies are described in U.S. Pat. No. 6,946,129 and U.S. Pat. No. 8,303,955.

Therefore in one preferred embodiment the agonistic CD40 antibody used in the combination therapy with an ANG-2 or an ANG-2/VEGF antibody is characterized by comprising (a) a heavy chain variable domain amino acid sequence of QVQLVQSGAEVKKPGASVKVSCKAS GYTFTGYYMHWVRQAPGQGLEWMGWINPDSGGTNYAQKFQGRVTMTR DTSISTAYMELNRLRSDDTAVYYCARDQPLGYCTNGVCSYFDYWGQGTL VTVSS (SEQ ID NO: 5) (which corresponds to SEQ ID NO: 42 of U.S. Pat. No. 7,338,660) (b) a light chain variable domain amino acid sequence of DIQMTQSPSSVSASVGDRVTITCRASQGIYSWLAWYQQKPGKAPNLLIYTA STLQSGVPSRFSGSGSGTDFTLTISSLQPEDFATYYCQQANIFPLTFGGGTKV EIK (SEQ ID NO: 6) (which corresponds to SEQ ID NO: 44 of U.S. Pat. No. 7,338,660); and/or having the heavy chain variable domain and light chain variable domain amino acid sequences of the antibody produced by hybridoma 21.4.1 having American Type Culture Collection (ATCC) accession number PTA-3605. Dacetuzumab and other humanized S2C6 antibodies are described in U.S. Pat. No. 6,946,129 and U.S. Pat. No. 8,303,955.

In one preferred embodiment the agonistic CD40 antibody used in the combination therapy with an ANG-2 or an ANG-2/VEGF antibody is a humanized S2C6 antibody. A humanized S2C6 antibody is e.g. based on the CDR1, 2 and 3 of the heavy and light chain variable domain of murine mAB S2C6 (deposited with the ATCC as PTA-110). The CDR1, 2 and 3 of the heavy and light chain variable domain of murine mAB S2C6 is described and disclosed US 6,946,129. In one embodiment the agonist CD40 antibody is dacetuzumab. In one embodiment the agonist CD40 antibody is characterized by comprising (a) a heavy chain variable domain amino acid sequence of EVQLVESGGGLVQPGGSLRLSCAASGYSFTGYYIHWVRQAPGKGLEWVA RVIPNAGGTSYNQKFKGRFTLSVDNSKNTAYLQMNSLRAEDTAVYYCARE GIYWWGQGTLVTVS (SEQ ID NO: 7) (b) a light chain variable domain amino acid sequence of DIQMTQSPSSLSASVGDRVTITCRSSQSLVHSNGNTFLHW YQQKPGKAPKLLIYTVSNRFSGVPSRFSGSGSGTDFTLTISSLQPEDFAT YFCSQTTHVPWTFGQGTKVEIKR (SEQ ID NO: 8).

In one embodiment the agonist CD40 antibody is a human antibody. As used herein, the term “human antibody” means an antibody in which the variable and constant domain sequences are derived from human sequences. Human agonistic CD40 antibodies are described in detail in WO 03/040170 the entire disclosure of which is hereby incorporated by reference. Human antibodies provide a substantial advantage in the treatment methods of the present invention, as they are expected to minimize the immunogenic and allergic responses that are associated with use of non-human antibodies in human patients.

Other Exemplary human anti-CD40 antibodies useful for the present invention include antibodies having the amino acid sequences of antibodies designated 3.1.1, 3.1.1 H-A78T, 3.1.1 H-A78T-V88A-V97A, 7.1.2, 10.8.3, 15.1.1, 21.4.1, 21.2.1, 22.1.1, 22.1.1 H-C109A, 23.5.1, 23.25.1, 23.28.1, 23.28.1H-D16E, 23.29.1, 24.2.1, 3.1.1H-A78T-V88A-V97A/3.1.1L-L4M-L83V and 23.28.1L-C92A, described in WO03/040170 as well as an antibody comprising a CDR or variable region of any of the exemplary antibodies.

In certain embodiments, the cancer or tumor treatment inhibits cancer cell proliferation, inhibits or prevents an increase in tumor weight or volume, and/or causes a decrease in tumor weight or volume. In some embodiments, the cancer treatment prolongs patient survival. In certain embodiments, tumor growth is inhibited at least 50%, 55%, 60%, 65%, 70% or 75%, compared to those not treated. In some embodiments, the cancer or tumor is CD40 positive.

In the present invention the term “CD40L” or “CD154” as it alternatively known in the art includes all mammalian CD40L's, e.g., human, rat, non-human primate, murine as well as fragments, variants, oligomers, and conjugates thereof that bind to at least the corresponding mammalian CD40 polypeptide, e.g., human CD40. In the present invention the administered CD40L may comprise a CD40L polypeptide or a DNA encoding said CD40L polypeptide. Such CD40L polypeptides and DNAs include in particular native CD40L sequences and fragments, variants, and oligomers thereof as disclosed in Immunex U.S. Pat. No. 6,410,711; U.S. Pat. No. 6,391,637; U.S. Pat. No. 5,981,724; U.S. Pat. No. 5,961,974 and US published application No. 20040006006 all of which patents and application and the CD40L sequences disclosed therein are incorporated by reference in their entirety herein.

The CD40L polypeptide can be used as CD40 agonist according to the invention and includes in particular native CD40L sequences and fragments, variants, and oligomers thereof as disclosed in Immunex U.S. Pat. No. 6,410,711; U.S. Pat. No. 6,391,637; U.S. Pat. No. 5,981,724; U.S. Pat. No. 5,961,974 and US published application No. 20040006006 all of which patents and application and the CD40L sequences disclosed therein are incorporated by reference in their entirety herein.

The term “epitope” denotes a protein determinant of the antigen capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents.

The “variable domain” (light chain variable domain VL, heavy chain variable domain VH) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The terms “nucleic acid” or “nucleic acid molecule”, as used herein, are intended to include DNA molecules and RNA molecules. A nucleic acid molecule may be single-stranded or double-stranded, but preferably is double-stranded DNA.

The term “amino acid” as used within this application denotes the group of naturally occurring carboxy alpha-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The “Fc part” of an antibody is not involved directly in binding of an antibody to an antigen, but exhibit various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α, δ, ε, γ, and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al., Nature 282 (1979) 742-743; Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virology 75 (2001) 12161-12168; Morgan, A., et al., Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3.

In one embodiment the antibodies described herein are of human IgG class (i.e. of IgG1, IgG2, IgG3 or IgG4 subclass).

In a preferred embodiment the antibodies described herein are of human IgG1 subclass or of human IgG4 subclass. In one embodiment the described herein are of human IgG1 subclass. In one embodiment the antibodies described herein are of human IgG4 subclass.

In one embodiment the antibody according to the invention comprises an Fc part derived from human origin and preferably all other parts of the human constant regions. As used herein the term “Fc part derived from human origin” denotes a Fc part which is either a Fc part of a human antibody of the subclass IgG1, IgG2, IgG3 or IgG4, preferably a Fc part from human IgG1 subclass, a mutated Fc part from human IgG1 subclass (in one embodiment with a mutation on L234A+L235A), a Fc part from human IgG4 subclass or a mutated Fc part from human IgG4 subclass (in one embodiment with a mutation on S228P).

In one embodiment the antibody described herein is characterized in that the constant chains are of human origin. Such constant chains are well known in the state of the art and e.g. described by Kabat, E. A., (see e.g. Johnson, G. and Wu, T. T., Nucleic Acids Res. 28 (2000) 214-218).

The invention comprises a method for the treatment of a patient in need of therapy, characterized by administering to the patient a therapeutically effective amount of the anti-ANG2 antibody described herein with a therapeutically effective amount of a CD40 agonist (e.g. an agonistic CD40 antibody). The invention comprises the use of the anti-ANG2 antibody described herein with a CD40 agonist (e.g. an agonistic CD40 antibody) for the described therapy.

The antibodies described herein are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis).

Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger, E.-J. and Christensen, K., in Cytotechnology 30 (1999) 71-83, and by Schlaeger, E.-J., in J. Immunol. Methods 194 (1996) 191-199.

The heavy and light chain variable domains according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

In another aspect, the present invention provides a composition, e.g. a pharmaceutical composition, containing one or a combination of monoclonal antibodies, or the antigen-binding portion thereof, of the present invention, formulated together with a pharmaceutically acceptable carrier.

As used herein, “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion.

A composition of the present invention can be administered by a variety of methods known in the art. As will be appreciated by the skilled artisan, the route and/or mode of administration will vary depending upon the desired results.

Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient (effective amount). The selected dosage level will depend upon a variety of pharmacokinetic factors including the activity of the particular compositions of the present invention employed, or the ester, salt or amide thereof, the route of administration, the time of administration, the rate of excretion of the particular compound being employed, other drugs, compounds and/or materials used in combination with the particular compositions employed, the age, sex, weight, condition, general health and prior medical history of the patient being treated, and like factors well known in the medical arts.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “administered in combination with” or “co-administration”, “co-administering”, “combination therapy”, “ administered with” or “combination treatment” refer to the administration of the anti-ANG2 antibody as described herein, and the agonistic CD40 antibody as described herein e.g. as separate formulations/applications (or as one single formulation/application). The co-administration can be simultaneous or sequential in either order, wherein preferably there is a time period while both (or all) active agents simultaneously exert their biological activities. Said antibody and said further agent are co-administered either simultaneously or sequentially (e.g. intravenous (i.v.) through a continuous infusion. When both therapeutic agents are co-administered sequentially the dose is administered either on the same day in two separate administrations, or one of the agents is administered on day 1 and the second is co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time. The terms “co-administration” with respect to the maintenance doses of anti-ANG2 antibody and/or agonistic CD40 antibody mean that the maintenance doses can be either co-administered simultaneously, if the treatment cycle is appropriate for both drugs, e.g. every week. Or the further agent is e.g. administered e.g. every first to third day and said antibody is administered every week. Or the maintenance doses are co-administered sequentially, either within one or within several days.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

As used herein, the term “patient” or “subject” preferably refers to a human in need of treatment of cancer, or a precancerous condition or lesion. However, the term “patient” can also refer to non-human animals, e.g. mammals such as mice, dogs, cats, horses, cows, pigs, sheep and non-human primates, among others, that are in need of treatment.

The amount of co-administration and the timing of co-administration will depend on the type (species, gender, age, weight, etc.) and condition of the patient being treated and the severity of the disease or condition being treated. Said anti-ANG2 antibody and further agent are suitably co-administered to the patient at one time or over a series of treatments e.g. on the same day or on the day after.

Depending on the type and severity of the disease, about 0.1 mg/kg to 50 mg/kg (e.g. 0.1-20 mg/kg) of said anti-ANG2 antibody and/or agonistic CD40 antibody; is an initial candidate dosage for co-administration of both drugs to the patient The invention comprises the use of the antibodies according to the invention for the treatment of a patient suffering from cancer, especially from colon cancer, ovarian cancer, glioblastoma, gastric cancer, pancreatic cancer, breast cancer, lung cancer, hepatocellular cancer.

In addition to the anti-ANG2 antibody in combination with the agonistic CD40 antibody also a chemotherapeutic agent can be administered.

In one embodiment such additional chemotherapeutic agents, which may be administered with anti-ANG2 antibody as described herein and the agonistic CD40 antibody as described herein, include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-merca.rho.topurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza) and release of transcriptional repression (ATRA) therapies can also be combined with the antigen binding proteins. In one embodiment the chemotherapeutic agent is selected from the group consisting of taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio). In one embodiment, the additional chemotherapeutic agent is selected from 5-fluorouracil (5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

Specific examples of combination therapies with additional chemotherapeutic agents include, for instance, therapies taxanes (e.g., docetaxel or paclitaxel) or a modified paclitaxel (e.g., Abraxane or Opaxio), doxorubicin), capecitabine and/or bevacizumab (Avastin) for the treatment of breast cancer; therapies with carboplatin, oxaliplatin, cisplatin, paclitaxel, doxorubicin (or modified doxorubicin (Caelyx or Doxil)), or topotecan (Hycamtin) for ovarian cancer, the therapies with a multi-kinase inhibitor, MKI, (Sutent, Nexavar, or 706) and/or doxorubicin for treatment of kidney cancer; therapies with oxaliplatin, cisplatin and/or radiation for the treatment of squamous cell carcinoma; therapies with taxol and/or carboplatin for the treatment of lung cancer.

Therefore, in one embodiment the additional chemotherapeutic agent is selected from the group of taxanes (docetaxel or paclitaxel or a modified paclitaxel (Abraxane or Opaxio), doxorubicin, capecitabine and/or bevacizumab for the treatment of breast cancer.

In one embodiment of the anti-ANG2 antibody/agonistic CD40 antibody combination therapy, no additional chemotherapeutic agents are administered.

The invention comprises also a method for the treatment of a patient suffering from such disease.

The invention further provides a method for the manufacture of a pharmaceutical composition comprising an effective amount of an antibody according to the invention together with a pharmaceutically acceptable carrier and the use of the antibody according to the invention for such a method.

The invention further provides the use of an antibody according to the invention in an effective amount for the manufacture of a pharmaceutical agent, preferably together with a pharmaceutically acceptable carrier, for the treatment of a patient suffering from cancer.

The invention also provides the use of an antibody according to the invention in an effective amount for the manufacture of a pharmaceutical agent, preferably together with a pharmaceutically acceptable carrier, for the treatment of a patient suffering from cancer.

In the following some specific embodiments of the invention are listed:
1. An antibody that binds to human angiopoietin 2 (ANG-2) wherein the antibody is administered in combination with CD40 agonist a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).
2. An antibody that binds to human angiopoietin 2 (ANG-2) wherein the antibody is administered in combination with CD40 agonist a) for use in treating or delaying progression of cancer, b) for use in prolonging the survival of a patient suffering from cancer.
3. The anti-ANG2 antibody for use according to embodiment 1 or 2, wherein the anti-ANG2 antibody is a monoclonal antibody.
4. The anti-ANG2 antibody for use according to any one of the preceding embodiments, wherein the anti-ANG2 antibody is human or humanized.
5. The anti-ANG2 antibody for use according to any one of the preceding embodiments, wherein the anti-ANG2 antibody specifically binds to human ANG2 with a K_{D} value of less than 1.0×10⁻⁸ mol/l, as determined by surface plasmon resonance (Biacore™).
6. The anti-ANG2 antibody for use according to any one of the preceding embodiments, wherein the anti-ANG2 antibody is an IgG antibody.
7. The anti-ANG2 antibody for use according to any one of the preceding embodiments, wherein the anti-ANG2 antibody inhibits the interaction of human ANG-2 with TIE2 receptor with an IC50 of 15 nM or less.
8. The anti-ANG2 antibody for use according to any one of the preceding embodiments, wherein the CD40 agonist is an agonistic CD40 antibody or an agonistic CD40L polypeptide.
9. The anti-ANG2 antibody for use according to any one of the preceding embodiments, wherein the CD40 agonist is an agonistic CD40 antibody.
10. The antibody that binds to human ANG-2 according to embodiment 9,
   i) wherein the anti-ANG2 antibody comprises
   (a) a heavy chain variable domain amino acid sequence of SEQ ID NO:1 and a light chain variable domain amino acid sequence of SEQ ID NO:2; or
   (b) a heavy chain variable domain amino acid sequence of SEQ ID NO:3 and a light chain variable domain amino acid sequence of SEQ ID NO:4; and
   ii) wherein the agonistic CD40 antibody comprises
   (a) a heavy chain variable domain amino acid sequence of SEQ ID NO: 5 and a light chain variable domain amino acid sequence of SEQ ID NO: 6; or
   (b) a heavy chain variable domain amino acid sequence of SEQ ID NO: 7 and a light chain variable domain amino acid sequence of SEQ ID NO: 8;
11. The antibody that binds to human ANG-2 according to embodiment 9,
   i) wherein the anti-ANG2 antibody comprises
   a heavy chain variable domain amino acid sequence of SEQ ID NO:1 and a light chain variable domain amino acid sequence of SEQ ID NO:2; or and
   ii) wherein the agonistic CD40 antibody comprises
   a heavy chain variable domain amino acid sequence of SEQ ID NO: 5 and a light chain variable domain amino acid sequence of SEQ ID NO: 6.
12. The anti-ANG2 antibody for use according to embodiment 9, wherein the anti-ANG2 antibody is a bispecific antibody that binds to human ANG-2 and that binds to human VEGF.
13. The antibody that binds to human ANG-2 according embodiment 10,
   i) wherein the bispecific antibody that binds to human ANG-2 and that binds to human VEGF comprises
   a heavy chain variable domain amino acid sequence of SEQ ID NO:1 and a light chain variable domain amino acid sequence of SEQ ID NO:2; and
   a heavy chain variable domain amino acid sequence of SEQ ID NO:9 and a light chain variable domain amino acid sequence of SEQ ID NO:10; and
   ii) wherein the agonistic CD40 antibody comprises
   a heavy chain variable domain amino acid sequence of SEQ ID NO: 5 and a light chain variable domain amino acid sequence of SEQ ID NO: 6.
14. The antibody that binds to human ANG-2 according embodiment 10,
   i) wherein the bispecific antibody that binds to human ANG-2 and that binds to human VEGF comprises the amino acid sequences of SEQ ID NO: 11, of SEQ ID NO: 12, of SEQ ID NO: 13, and of SEQ ID NO: 14; and
   ii) wherein the agonistic CD40 antibody comprises
   a heavy chain variable domain amino acid sequence of SEQ ID NO: 5 and a light chain variable domain amino acid sequence of SEQ ID NO: 6.
15. An antibody that binds to human angiopoietin 2 (ANG-2) for use according to any one of the preceding embodiments, wherein the cancer is lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma or lymphocytic leukemia.
16. An antibody that binds to human angiopoietin 2 (ANG-2) for use according to embodiment 15, wherein the cancer comprises a solid tumor.
17. An antibody that binds to human angiopoietin 2 (ANG-2) for use according to embodiment 15 or 16, wherein the cancer is colon cancer, ovarian cancer, glioblastoma, gastric cancer, pancreatic cancer, breast cancer, lung cancer, hepatocellular cancer.
18. The antibody that binds to human angiopoietin 2 (ANG-2) for use according to any one of the preceding embodiments, wherein in the method of treatment, the anti-ANG2 antibody and the CD40 agonist are administered to the subject simultaneously, separately or sequentially.

Description of the Amino Acid Sequences
SEQ ID NO: 1 variable heavy chain domain VH of <ANG-2> E6Q
SEQ ID NO: 2 variable light chain domain VL of <ANG-2> E6Q
SEQ ID NO: 3 variable heavy chain domain VH of <ANG-2> Ang2i_LC06
SEQ ID NO: 4 variable light chain domain VL of <ANG-2> Ang2i_LC06
SEQ ID NO: 5 variable heavy chain domain VH of CP-870,893 (antibody 21.4.1 of U.S. Pat. No. 7,338,660)
SEQ ID NO: 6 variable light chain domain VL of CP-870,893 (antibody 21.4.1 of U.S. Pat. No. 7,338,660)
SEQ ID NO: 7 humanized S2C6 heavy chain variable domain VH variant
SEQ ID NO: 8 humanized S2C6 light chain variable domain VL variant
SEQ ID NO: 9 variable heavy chain domain VH of <VEGF> bevacizumab
SEQ ID NO: 10 variable light chain domain VL of <VEGF> bevacizumab
SEQ ID NO: 11 Bispecific ANG2/VEGF antibody XMab1-<VEGF> light chain
SEQ ID NO: 12 Bispecific ANG2/VEGF antibody XMab1-<ANG2> light chain
SEQ ID NO: 13 Bispecific ANG2/VEGF antibody XMab1-<VEGF> heavy chain
SEQ ID NO: 14 Bispecific ANG2/VEGF antibody XMab1-<ANG2> heavy chain
SEQ ID NO: 15 Human angiopoietin-2 (ANG-2)
SEQ ID NO: 16 Human vascular endothelial growth factor (VEGF)
SEQ ID NO: 17 variable heavy chain domain VH of <VEGF> B20-4.1
SEQ ID NO: 18 variable light chain domain VL of <VEGF> B20-4.1

In the following embodiments of the invention are described:
1. An antibody that binds to human angiopoietin 2 (ANG-2) wherein the antibody is administered in combination with a CD40 agonist a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).
2. An antibody that binds to human angiopoietin 2 (ANG-2) wherein the antibody is administered in combination with a CD40 agonist a) for use in treating or delaying progression of cancer, b) for use in prolonging the survival of a patient suffering from cancer. 3. Use of a combination of
i) an antibody that binds to human angiopoietin 2 (ANG-2), and
   ii) a CD40 agonist
   for the manufacture of a medicament
   a) for use in treating or delaying progression of cancer, or b) for use in prolonging the survival of a patient suffering from cancer, or c) for use in stimulating an immune response or function, such as T cell activity (in one embodiment CD8 effector T cell activity) or macrophage activity (in one embodiment CD40-activated macrophage activity), or d) for use in rendering a cancer susceptible for the treatment with an antibody that binds to human angiopoietin 2 (ANG-2).
4. Use of a combination of
   i) an antibody that binds to human angiopoietin 2 (ANG-2), and
   ii) a CD40 agonist
   for the manufacture of a medicament
   a) for use in treating or delaying progression of cancer, b) for use in prolonging the survival of a patient suffering from cancer.
5. The anti-ANG2 antibody for use or the use according to any one of the preceding embodiments, wherein the anti-ANG2 antibody is human or humanized.
6. The anti-ANG2 antibody for use or the use according to any one of the preceding embodiments, wherein the anti-ANG2 antibody specifically binds to human ANG2 with a K_{D} value of less than 1.0×10⁻⁸ mol/l, as determined by surface plasmon resonance (Biacore™).
7. The anti-ANG2 antibody for use or the use according to any one of the preceding embodiments, wherein the anti-ANG2 antibody is an IgG antibody.
8. The anti-ANG2 antibody for use or the use according to any one of the preceding embodiments, wherein the anti-ANG2 antibody inhibits the interaction of human ANG-2 with TIE2 receptor with an IC50 of 15 nM or less.
9. The anti-ANG2 antibody for use or the use according to any one of the preceding embodiments, wherein the CD40 agonist is an agonistic CD40 antibody or an agonistic CD40L polypeptide.
10. The anti-ANG2 antibody for use or the use according to any one of the preceding embodiments, wherein the CD40 agonist is an agonistic CD40 antibody.
11. The antibody that binds to human ANG-2 or the use according to embodiment 10,
   i) wherein the anti-ANG2 antibody comprises
   (a) a heavy chain variable domain amino acid sequence of SEQ ID NO:1 and a light chain variable domain amino acid sequence of SEQ ID NO:2; or
   (b) a heavy chain variable domain amino acid sequence of SEQ ID NO:3 and a light chain variable domain amino acid sequence of SEQ ID NO:4; and
   ii) wherein the agonistic CD40 antibody comprises
   (a) a heavy chain variable domain amino acid sequence of SEQ ID NO: 5 and a light chain variable domain amino acid sequence of SEQ ID NO: 6; or
   (b) a heavy chain variable domain amino acid sequence of SEQ ID NO: 7 and a light chain variable domain amino acid sequence of SEQ ID NO: 8;
12. The antibody that binds to human ANG-2 or the use according to embodiment 10,
   i) wherein the anti-ANG2 antibody comprises
   a heavy chain variable domain amino acid sequence of SEQ ID NO:1 and a light chain variable domain amino acid sequence of SEQ ID NO:2; or and
   ii) wherein the agonistic CD40 antibody comprises
   a heavy chain variable domain amino acid sequence of SEQ ID NO: 5 and a light chain variable domain amino acid sequence of SEQ ID NO: 6; or
13. The anti-ANG2 antibody for use or the use according to embodiment 10, wherein the anti-ANG2 antibody is a bispecific antibody that binds to human ANG-2 and that binds to human VEGF.
14. The anti-ANG2 antibody for use or the use according to embodiment 10, wherein additionally an antibody that binds to human VEGF is administered in combination or is used in the combination with the CD40 agonist.
15. The antibody that binds to human ANG-2 or the use according to embodiment 13,
   i) wherein the bispecific antibody that binds to human ANG-2 and that binds to human VEGF comprises
   a heavy chain variable domain amino acid sequence of SEQ ID NO:1 and a light chain variable domain amino acid sequence of SEQ ID NO:2; and
   a heavy chain variable domain amino acid sequence of SEQ ID NO:9 and a light chain variable domain amino acid sequence of SEQ ID NO:10; and
   ii) wherein the agonistic CD40 antibody comprises
   a heavy chain variable domain amino acid sequence of SEQ ID NO: 5 and a light chain variable domain amino acid sequence of SEQ ID NO: 6.
16. The antibody that binds to human ANG-2 or the use according to embodiment 13,
   i) wherein the bispecific antibody that binds to human ANG-2 and that binds to human VEGF comprises the amino acid sequences of SEQ ID NO: 11, of SEQ ID NO: 12, of SEQ ID NO: 13, and of SEQ ID NO: 14; and
   ii) wherein the agonistic CD40 antibody comprises
   a heavy chain variable domain amino acid sequence of SEQ ID NO: 5 and a light chain variable domain amino acid sequence of SEQ ID NO: 6.
17. The antibody that binds to human ANG-2 or the use according to embodiment 14,
   i) wherein the antibody that binds to human VEGF comprises
   a heavy chain variable domain amino acid sequence of SEQ ID NO:9 and a light chain variable domain amino acid sequence of SEQ ID NO:10; and
   ii) wherein the agonistic CD40 antibody comprises
   a heavy chain variable domain amino acid sequence of SEQ ID NO: 5 and a light chain variable domain amino acid sequence of SEQ ID NO: 6.
18. An antibody that binds to human angiopoietin 2 (ANG-2) for use or the use according to any one of the preceding embodiments, wherein the cancer is lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma or lymphocytic leukemia.
19. An antibody that binds to human angiopoietin 2 (ANG-2) for use or the use according to embodiment 18, wherein the cancer comprises a solid tumor.
20. The antibody or use according to embodiments 18 or 19, wherein the cancer is further characterized by ANG-2 expression or overexpression.
21. An antibody that binds to human angiopoietin 2 (ANG-2) for use according to any one of embodiments 18 to 20, wherein the cancer is colon cancer, ovarian cancer, glioblastoma, gastric cancer, pancreatic cancer, breast cancer, lung cancer, hepatocellular cancer.
22. The antibody that binds to human angiopoietin 2 (ANG-2) for use according to any one of the preceding embodiments, wherein in the method of treatment, the anti-ANG2 antibody and the CD40 agonist are administered to the subject simultaneously, separately or sequentially.

### EXAMPLE 1

Inhibition of the Interaction of Human ANG-2 with TIE2 Receptor (Experiment A)

Blocking of human ANG-2/human Tie2 interaction was shown by receptor interaction ELISA. 384-well Maxisorp plates (Nunc) were coated with 0.5 μg/ml human Tie2 (R&D Systems, UK, Cat. No. 313-TI or in house produced material) for 2 h at room temperature and blocked with PBS supplemented with 0.2% Tween-20 and 2% BSA (Roche Diagnostics GmbH, DE) for 1 h at room temperature under shaking In the meantime, Dilutions of purified antibodies in PBS were incubated together with 0.2 μg/ml huAngiopoietin-1/2 (R&D Systems #923-AN/CF, R&D Systems, UK, Cat. No. 623-AN or in house produced material) for 1 hour at RT. After washing a mixture of 0.5 μg/ml biotinylated anti-Angiopoietin-1/2 clone (R&D Systems #BAF923, BAM0981 R&D Systems, UK) and 1:3000 diluted streptavidin HRP (Roche Diagnostics GmbH, DE, Cat. No. 11089153001) was added for 1 h. Thereafter the plates were washed 6 times with PBST. Plates were developed with freshly prepared ABTS reagent (Roche Diagnostics GmbH, DE, buffer #204 530 001, tablets #11 112 422 001) for 30 minutes at RT. Absorbance was measured at 405 nm.

The obtained inhibitory concentrations are summarized in the following table.

| | ANG2/Tie2 |
|---|---|
| Antibody | interaction ELISA |
| Ang2i-LC06 | 0.1 nM |

Inhibition of the Interaction of Human ANG-2 with TIE2 Receptor (Experiment B)

The interaction ELISA was performed on 384 well microtiter plates (MicroCoat, DE, Cat. No. 464718) at RT. After each incubation step plates were washed 3 times with PBST. ELISA plates were coated with 5 μg/ml Tie-2 protein for 1 hour (h). Thereafter the wells were blocked with PBS supplemented with 0.2% Tween-20 and 2% BSA (Roche Diagnostics GmbH, DE) for 1 h. Dilutions of purified bispecific Xmab antibodies in PBS were incubated together with 0.2 μg/ml huAngiopoietin-2 (R&D Systems, UK, Cat. No. 623-AN) for 1 h at RT. After washing a mixture of 0.5 μg/ml biotinylated anti-Angiopoietin-2 clone BAM0981 (R&D Systems, UK) and 1:3000 diluted streptavidin HRP (Roche Diagnostics GmbH, DE, Cat. No. 11089153001) was added for 1 h. Thereafter the plates were washed 3 times with PBST. Plates are developed with freshly prepared ABTS reagent (Roche Diagnostics GmbH, DE, buffer #204 530 001, tablets #11 112 422 001) for 30 minutes at RT. Absorbance was measured at 405 nm and the IC50 was determined.

XMab1, a bispecific antibody that binds to human ANG2 and to human VEGF (see WO2011/117329 and sequences SEQ ID NOs:11-14, showed an inhibition of ANG-2 binding to Tie-2 (ANG2/Tie2 receptor interaction inhibition) with an IC50 of 12 nM.

### EXAMPLE 2

In Vivo Anti-Tumor Efficacy of Anti-ANG2 Antibodies in Combination with an Agonistic CD40 Antibody (Delaying of Progression (Tumor Growth) of Cancer and Prolonging the Survival of Patients Treated)

Methods

C57BL/6 mice were s.c. injected with 500.000 syngeneic MC-38 tumor cells and treated beginning day 16 when tumors reached a size of 40-60 mm³.

Mice were euthanized according to veterinary regulations when tumors reached a size of 1500 mm³ (endpoint). As anti-ANG2 antibody a monospecific IgG1 antibody based on the VH and VL of <ANG-2> Ang2i_LC06 (SEQ ID NOs:3-4) was used. As mouse-cross reactive surrogate VEGF antibody based on the VH and VL of <VEGF> B20-4.1 (SEQ ID NOs:17-18) was used instead of the VH and VL of <VEGF> bevacizumab (SEQ ID NOs:9-10), as bevacizumab is not mouse crossreactive. These antibodies were used either alone or in combination. For similar reasons instead of the agonistic CD40 antibody CP-870,893 (antibody 21.4.1 of U.S. Pat. No. 7,338,660) (see VH and VL of SEQ ID NOs:5-6) the mouse cross-reactive agonistic CD40 antibody Clone 1C10 (see Santos-Argumedo L. et al., Cell Immunol. 156 (1994) 272-285, Heath A W et al. Eur J Immunol 24 (1994) 1828-34; obtainable e.g. from Abnova Catalog #MAB5607) was used, specifically in aa mouse IgG1 format (which can be either generated from the variable regions VH and VL of Clone 1C10 in combination with a mouse IgG1 constant region or which is also obtainable from Rockefeller University). For comparison also the combination with monospecific anti-VEGF antibody <VEGF> B20-4.1 (SEQ ID NOs:17-18) was examined. However similar experiments with transgenic humanized mice or clinical trials can be conducted using agonistic CD40 antibody CP-870,893 (antibody 21.4.1 of U.S. Pat. No. 7,338,660) and the bispecific ANG2/VEGF antibody XMab1 ((SEQ ID NOs:11-14) expecting similar result based on the mechanism of action.

Treatments were applied according to the following schedule below at the indicated doses.

| | |
|---|---|
| Tumor cell injection | Starting at day 500.000 syngeneic |
| | MC-38 tumor cells injected and treatment |
| | started at day 16 when tumors reached a |
| | size of 40-60 mm³ |
| Agonistic CD40 antibody | 5 mg/kg i.p. at day 16, 18, 21 and 24 |
| alone | |
| Anti-ANG2 antibody | 10 mg/kg i.p. at day 16 and 21 |
| alone | |
| Anti-VEGF antibody | 10 mg/kg i.p at day 16 and 21 |
| alone | |
| anti-ANG2 antibody with | both at a dose of 10 mg/kg i.p at day 16 and 21 |
| anti-VEGF antibody | |
| (Anti-VEGF/Ang-2) | |
| Anti-ANG2 antibody | 10 mg/kg i.p. at day 16 and 21 |
| with | |
| agonistic CD40 antibody | 5 mg/kg i.p. at day 16, 18, 21 and 24 |
| anti-ANG2 antibody with | both at a dose of 10 mg/kg i.p. at |
| anti-VEGF antibody | day 16 and 21 |
| (Anti-VEGF/Ang-2) | |
| with | |
| agonistic CD40 antibody | 5 mg/kg i.p. at day 16, 18, 21 and 24 |
| Anti-VEGF antibody | 10 mg/kg i.p. at day 16 and 21 |
| with | |
| agonistic CD40 antibody | 5 mg/kg i.p. at day 16, 18, 21 and 24 |

Results

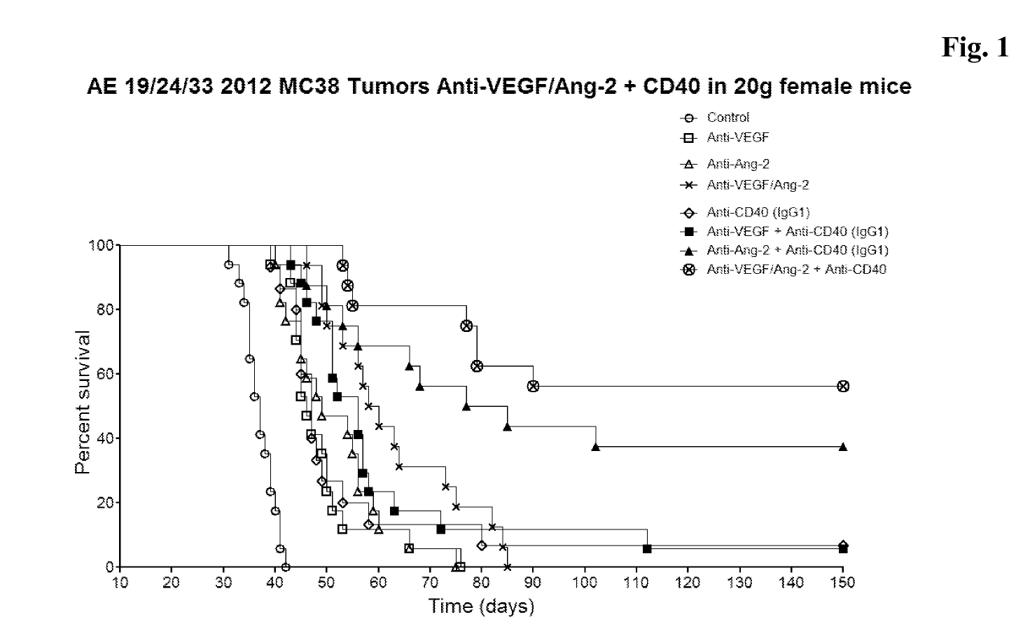

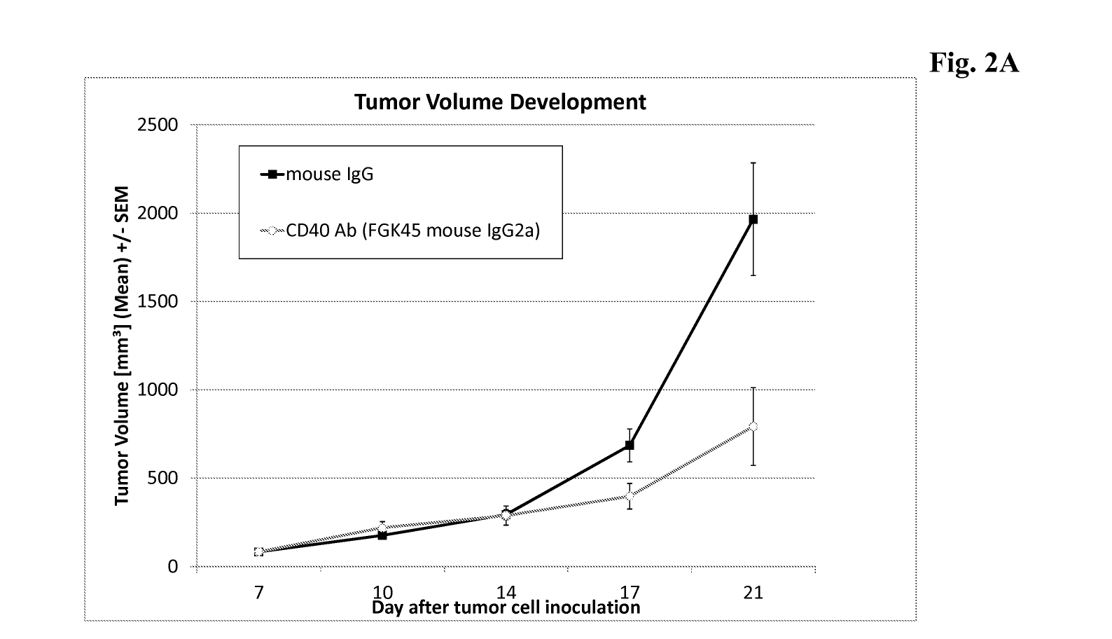

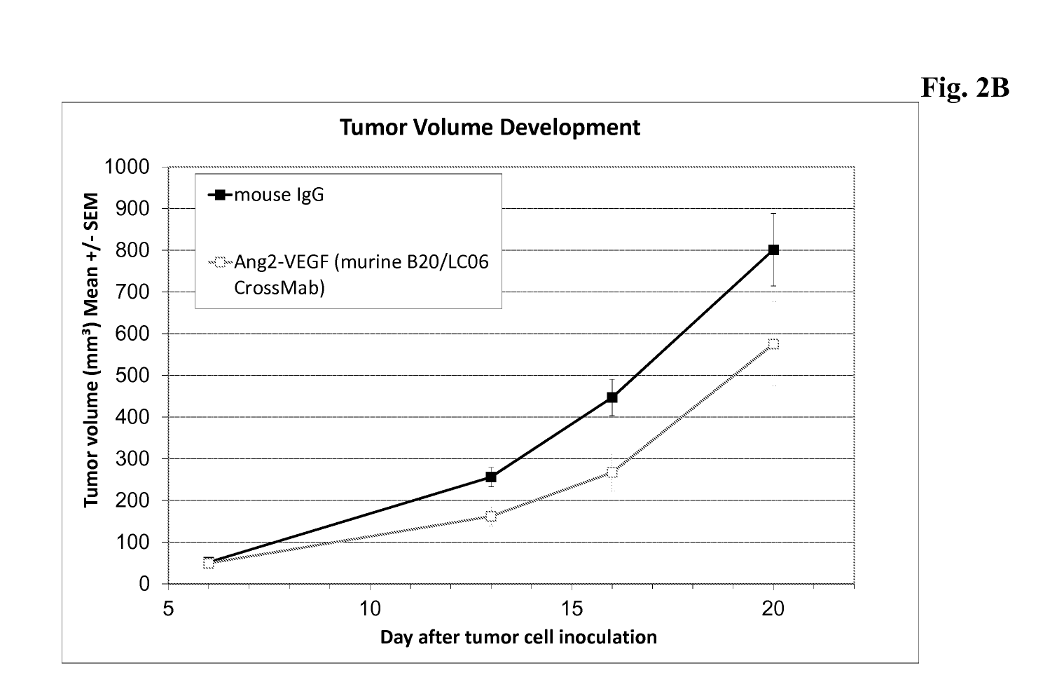

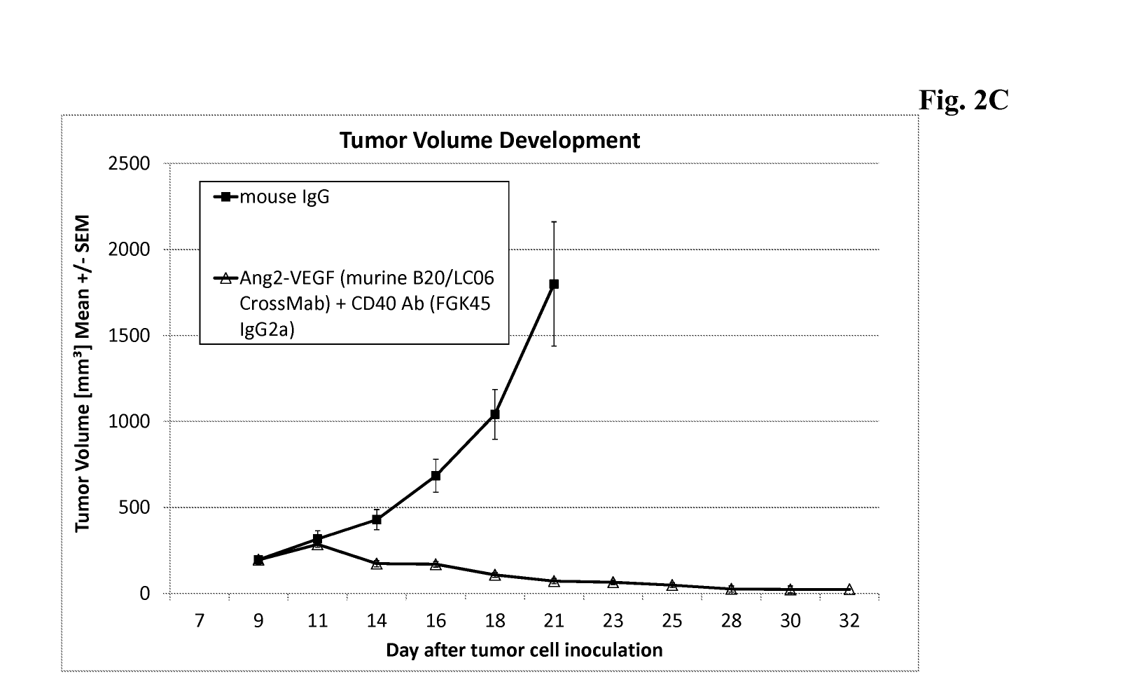

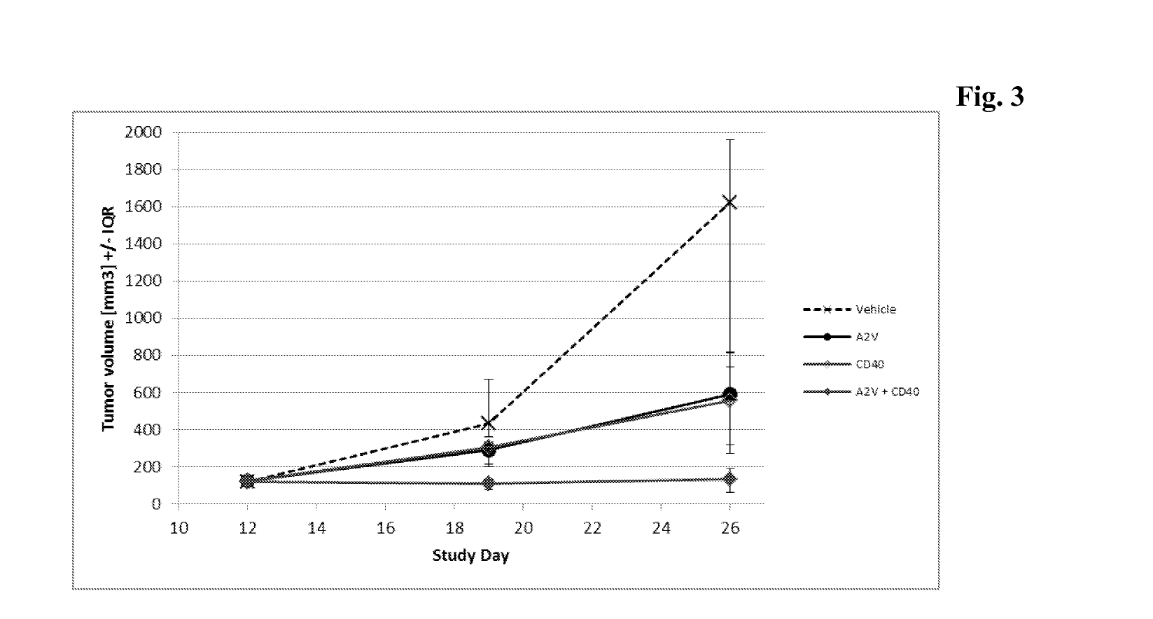

Results are shown in FIG. 1. The graph represents pooled data from 3 independent experiments. Mice were treated with the antibody combinations indicated in the figure.

Summary

Our data demonstrate that all used reagents do display some anti-tumor activity on their own. While the combination of an agonistic anti-CD40 antibody did only display small effects when combined with anti-VEGF antibody, the combination of agonistic CD40 antibody with anti-ANG2 antibodies (either monospecific ANG2 antibodies or the combination of anti-ANG2/anti-VEGF antibodies showed a strong synergistic effect on tumor cell growth, the delay of progression and the prolongation of survival. The strongest effect could be observed for the combination of agonistic CD40 antibody and the combination of anti-ANG2/anti-VEGF antibodies. A large proportion of tumor bearing mice were cured by the combined treatment with either anti-Ang-2 and anti-CD40 or the combination anti-ANG2/anti-VEGF antibodies (anti-VEGF/Ang-2) and anti-CD40. These combinations may therefore provide substantial therapeutic benefits for cancer patients.

### EXAMPLE 3

In Vivo Anti-Tumor Efficacy of Anti-ANG2 Antibodies (Bispecific) in Combination with an Agonistic CD40 Antibody in Subcutaneous Syngeneic MC38 Colon Carcinoma Model

Cells of the murine colorectal adenocarcinoma cell line MC-38 (obtained from Beckman Research Institute of the City of Hope, Calif., USA) were cultured in Dulbecco's Modified Eagle Medium (DMEM, PAN Biotech) supplemented with 10% FCS and 2 mM L-glutamine at 37° C. in a water saturated atmosphere at 5% CO2. At the day of inoculation, MC38 tumor cells were harvested with PBS from culture flasks and transferred into culture medium, centrifuged, washed once and re-suspended in PBS. For injection of cells, the final titer was adjusted to 1×107 cells/ml. Subsequently 100 μl of this suspension (1×106 cells) were inoculated subcutaneously into 6-10 weeks old female C57BL/6N mice. Groups of animals were treated with control antibodies (MOPC-21 (10 mg/kg i.p. once weekly) and/or 2A3 (100 μg i.p. once); Bio X Cell, West Lebanon), and with a bispecific anti-ANG2 antibody in combination with anti-CD40 monoclonal antibody FGK45 (agonist CD40 rat anti-mouse IgG2a mAb FGK45 (S. P. Schoenberger, et al, Nature, 393, 480 (1998), available from BioXcell) CD40 clone FGK. 45, 100 μg, i.p., once simultaneously with first dose of bispecific ANG2/VEGF antibody). the bispecific was a bispecific ANG2/VEGF antibody based on the VH and VL of <ANG-2> E6Q (SEQ ID NOs:1-2) for the ANG2 binding arm. As mouse-cross reactive surrogate VEGF binding arm the VH and VL of <VEGF> B20-4.1 (SEQ ID NOs:17-18) for the bispecific antibody were used instead of the VH and VL of <VEGF> bevacizumab (SEQ ID NOs:9-10). The structure, however was the same as that for XMab1 (only VH/VLs <VEGF> were exchanged for the mouse cross-reactive ones of B20-4.1))

Treatment started after tumors were established and had reached an average size of 50 to 80 mm3 for monotherapy or 200 mm3 for combination. Tumor volume was measured twice a week and animal weights were monitored in parallel. Results are shown in FIG. 7. Combination of bispecific ANG2/VEGF antibody with agonistic CD40 mAb shows improved anti-tumor efficacy over monotherapies in syngenic MC38 mouse adenocarcinoma model with tumors going in regression at day 21 (with TGI >100%). At day 32, 5 out of 10 animals were tumor free.

| | TGI | Study |
|---|---|---|
| Agonistic | 62% (day 21) | CSF1R_Pz_MC38_008 |
| CD40 | | |
| antibody | | |
| Bispecific | 30% (day 20) | CSF1R_Pz_MC38_009 |
| Ang2/ | | |
| VEGF | | |
| antibody | | |
| Combination | >100% (day 21) | CSF1R_Pz_MC38_011 |
| of | | |
| Agonistic | | |
| CD40 | | |
| antibody and | | |
| Bispecific | | |
| Ang2/ | | |
| VEGF | | |
| antibody | | |

### EXAMPLE 4

Anti-Tumor Efficacy of the Ang2/VEGF Antibody (CrossMab; LC06/B20.4.1) and the CD40 Antibody (FGK4.5; iTME-0004-0005) Alone or in Combination in the Syngeneic CT26WT Model in Female Balb/c Mice

Experimental Procedures

Test Agents

The bispecific ANG2/VEGF antibody based on the VH and VL of <ANG-2> E6Q (SEQ ID NOs:1-2) for the ANG2 binding arm. As mouse-cross reactive surrogate VEGF binding arm the VH and VL of <VEGF> B20-4.1 (SEQ ID NOs:17-18) for the bispecific antibody were used instead of the VH and VL of <VEGF> bevacizumab (SEQ ID NOs:9-10). The structure, however was the same as that for XMab1 (only VH/VLs <VEGF> were exchanged for the mouse cross-reactive ones of B20-4.1)). The bispecific ANG2/VEGF antibody was generated at Roche Diagnostics GmbH, Penzberg, Germany. The CD40 antibody (FGK4.5; iTME-0004-0005) was obtained from Adipogen/Biomol.

Antibody buffer included 20 mM histidine and 140 mM sodium chloride (pH 6.0). Antibody solutions were diluted appropriately in the above mentioned buffer from stock prior to administrations.

Cell Lines and Culture Conditions

The murine CT26WT cell line was routinely cultured in RPMI 1640 supplemented with 10% fetal bovine serum (PAA Laboratories, Austria) and 2 mM L-glutamine at 37° C. in a water-saturated atmosphere at 5% CO2.

Animals

Female Balb/c mice aged 6-7 weeks at arrival (purchased from Charles River, Sulzfeld, Germany) were maintained under specific-pathogen-free condition with daily cycles of 12 h light/12 h darkness according to committed guidelines (GV-Solas; Felasa; TierschG). Experimental study protocol was reviewed and approved by local government (Regierung von Oberbayern; registration no. 55.2-1-54-2531.2-32-10). After arrival animals were maintained in the animal facility for one week to get accustomed to new environment and for observation. Continuous health monitoring was carried out on regular basis. Diet food (Altromin) and water (filtered) were provided ad libitum.

Monitoring

Animals were controlled daily for clinical symptoms and detection of adverse effects. For monitoring throughout the experiment body weight of animals was documented two times weekly and tumor volume was measured by caliper after randomization.

Treatment of Animals

Animal treatment was started at the day of randomization at a tumor volume of about 130 mm³ 12 days after tumor cell inoculation. Following dosages, route of administration and treatment schedules have been applied:

TABLE 1
Applied dosages, route of administration and treatment schedules

| | Dose | Route of | Treatment |
|---|---|---|---|
| Compound | (mg/kg) | administration | schedule |
| Histidin buffer | — | IP | once weekly ×5 |
| RO6872840 | 10 | IP | once weekly ×2 |
| Crossmab | | | (Day 12/19) |
| (LC06/B20.4.1) | | | |
| CD40 | 100 μg/mouse | IP | Day 12 |
| (FGK4.5 | | | |
| iTME-0004-0005) | | | |
| CD40 + | 100 μg/mouse + | IP | Day 12 + |
| LC06/B20.4.1 | 10 | | once weekly ×2 |
| | | | (Day 12/19) |

1.-15. (canceled)

16. A method of treating a patient suffering from cancer comprising administering to said patient an antibody that specifically binds to human angiopoietin 2 (ANG-2) in combination with a CD40 agonist.

17. The method of claim 16, wherein the anti-ANG2 antibody is human or humanized.

18. The method of claim 17, wherein the anti-ANG2 antibody specifically binds to human ANG2 with a KD value of less than 1.0×10-8 mol/l, as determined by surface plasmon resonance (Biacore™).

19. The method of claim 16, wherein the anti-ANG2 antibody is an IgG antibody.

20. The method of claim 16, wherein the anti-ANG2 antibody inhibits the interaction of human ANG-2 with TIE2 receptor with an IC50 of 15 nM or less.

21. The method of claim 16, wherein the CD40 agonist is an agonistic CD40 antibody or an agonistic CD40L polypeptide.

22. The method of claim 21, wherein the CD40 agonist is an agonistic CD40 antibody.

23. The method of claim 16, wherein the anti-ANG2 antibody comprises:
(a) a heavy chain variable domain amino acid sequence of SEQ ID NO:1 and a light chain variable domain amino acid sequence of SEQ ID NO:2; or
(b) a heavy chain variable domain amino acid sequence of SEQ ID NO:3 and a light chain variable domain amino acid sequence of SEQ ID NO:4; and
wherein the agonistic CD40 antibody comprises:
(a) a heavy chain variable domain amino acid sequence of SEQ ID NO: 5 and a light chain variable domain amino acid sequence of SEQ ID NO: 6; or
(b) a heavy chain variable domain amino acid sequence of SEQ ID NO: 7 and a light chain variable domain amino acid sequence of SEQ ID NO: 8.

24. The method of claim 23, wherein the anti-ANG2 antibody comprises a heavy chain variable domain amino acid sequence of SEQ ID NO:1 and a light chain variable domain amino acid sequence of SEQ ID NO:2; and wherein the agonistic CD40 antibody comprises a heavy chain variable domain amino acid sequence of SEQ ID NO: 5 and a light chain variable domain amino acid sequence of SEQ ID NO: 6.

25. The method of claim 16, wherein the anti-ANG2 antibody is a bispecific antibody that specifically binds to human ANG-2 and that specifically binds to human VEGF.

26. The method of claim 25, wherein the bispecific antibody comprises an anti-ANG2 antibody arm comprising a heavy chain variable domain amino acid sequence of SEQ ID NO:1 and a light chain variable domain amino acid sequence of SEQ ID NO:2, and an anti-VEGF antibody arm comprising a heavy chain variable domain amino acid sequence of SEQ ID NO:9 and a light chain variable domain amino acid sequence of SEQ ID NO:10; and further wherein the agonistic CD40 agonist is an antibody comprising a heavy chain variable domain amino acid sequence of SEQ ID NO: 5 and a light chain variable domain amino acid sequence of SEQ ID NO: 6.

27. The method of claim 25, wherein the bispecific antibody comprises a light chain amino acid sequence of SEQ ID NO: 11 or 12 and a heavy chain amino acid sequence of SEQ ID NO: 13 or 14; and
wherein the CD40 agonist is an antibody comprising a heavy chain variable domain amino acid sequence of SEQ ID NO: 5 and a light chain variable domain amino acid sequence of SEQ ID NO: 6.

28. The method of claim 16, wherein the cancer is lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma or lymphocytic leukemia.

29. The method of claim 28, wherein the cancer is further characterized by ANG-2 expression or overexpression.

30. The method of claim 16, wherein the method delays the progression of cancer.

31. The method of claim 16, wherein the method prolongs the survival of a patient.

32. The method of claim 16, wherein the method stimulates an immune response or function.

33. The method of claim 32, wherein the stimulation of the immune response or function is a T cell activity or a macrophage activity.

34. The method of claim 33, wherein the T cell activity is a CD8+ T cell activity.

35. The method of claim 33, wherein the macrophage activity is a CD40-activated macrophage activity.

36. The method of claim 16, wherein the method renders the cancer susceptible for the treatment with the antibody that specifically binds to human ANG-2.


SEQUENCE LISTING (18 sequences)

SEQ ID NO: 1 - protein, 128 aa, Artificial
  variable heavy chain domain VH of <ANG-2> E6Q
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVS

SEQ ID NO: 2 - protein, 108 aa, Artificial
  variable light chain domain VL of < ANG-2> E6Q
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVL

SEQ ID NO: 3 - protein, 129 aa, Artificial
  variable heavy chain domain VH of <ANG-2> Ang2i_LC06
QVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 4 - protein, 110 aa, Artificial
  variable light chain domain VL of < ANG-2> Ang2i_LC06
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLGQ

SEQ ID NO: 5 - protein, 126 aa, Homo sapiens
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 6 - protein, 107 aa, Homo sapiens
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 7 - protein, 113 aa, Artificial
  humanized S2C6 heavy chain variabel domain variant
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVS

SEQ ID NO: 8 - protein, 113 aa, Artificial
  humanized S2C6 light chain variabel domain variant
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

SEQ ID NO: 9 - protein, 123 aa, Artificial [sequence omitted]

SEQ ID NO: 10 - protein, 107 aa, Artificial [sequence omitted]

SEQ ID NO: 11 - protein, 214 aa, Artificial
  XMab1 -<VEGF> light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 213 aa, Artificial
  XMab1 -<ANG2> light chain
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 453 aa, Artificial
  XMab1 -<VEGF> heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 463 aa, Artificial
  XMab1 -<ANG2> heavy chain
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 498 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADFSG

SEQ ID NO: 16 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 17 - protein, 117 aa, Artificial
  variable heavy chain domain VH of <VEGF> . B20-4.1
EVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLV

SEQ ID NO: 18 - protein, 108 aa, Artificial
  variable light chain domain VL of <VEGF> B20-4.1
DIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIKR